(19) United States
(12) Patent Application Publication
(10) Pub. No.: US 2023/0059066 A1
(43) Pub. Date: Feb. 23, 2023

(54) Pudendal Nerve Burst Stimulation for Bladder Control

(71) Applicants: Galvani Bioelectronics Limited, Brentford, Middlesex (GB); Duke University, Durham, NC (US)

(72) Inventors: Arun Sridhar, Brentford (GB); Philip Milliken, Brentford (GB); Christopher Lawrence Langdale, Apex, NC (US); Warren Murray Grill, Chapel Hill, NC (US); James Arthur Hokanson, Morrisville, NC (US)

(21) Appl. No.: 17/782,559

(22) PCT Filed: Dec. 9, 2020
(86) PCT No.: PCT/US2020/063967

§ 371 Date: Jun. 3, 2022

Related U.S. Application Data
(60) Provisional application No. 62/946,596, filed on Dec. 11, 2019.

(51) Int. Cl. A61N 1/36

(52) U.S. Cl. CPC A61N 1/3606; A61N 1/36171; A61N 1/36178; A61N 1/36157

(57) ABSTRACT

The present disclosure provides an apparatus for stimulating neural activity in a pudendal nerve of a subject, the apparatus comprising of: at least one primary electrode configured to apply a first electrical signal to said nerve; and a controller coupled to said primary electrode (s) and controlling the first electrical signal to be applied thereby, wherein said controller is configured to cause said at least one primary electrode to apply said first electrical signal that stimulates neural activity in the pudendal nerve to improve bladder function, and wherein the first electrical signal is applied in a burst pattern.

Bladder function is comprised of two phases: a filling phase (urine storage) and a voiding phase (urine evacuation) and efficient bladder function involves control of these phases mediated by continence and micturition reflexes accomplished through coordinated sympathetic, parasympathetic and somatic neural activity [Beckel and Holstege Neurophysiology of the Lower Urinary Tract, in Urinary Tract (2011) Springer Berlin Heidelberg, 149-169]. In bladder dysfunction (such as over-active bladder (OAB), underactive bladder (UAB), or urinary retention), one or more of these functions is disrupted, leading to symptoms including urinary urgency, frequency, urgency incontinence, nocturia, sensation of incomplete emptying, straining to void, and recurrent infections. These symptoms often fail to improve following pharmacological treatment alone (Izett et al. Minerva Ginecol. 2017 June; 69(3):269-285; McDonnell B and Birder, L A, Version 1. F1000Res. 2017; 6: 2148).

Pudendal nerve stimulation is a promising therapeutic option for treatment of bladder dysfunction symptoms, though it remains unclear how to optimally stimulate the pudendal nerve to reduce the symptoms of bladder dysfunction.

WO2017/066572 describes stimulation of the pudendal nerve with a high intensity electrical signal to improve bladder capacity and burst stimulation of the pudendal motor branch to promote voiding.

### SUMMARY OF THE INVENTION

The inventors have investigated electrical stimulation of the pudendal nerve, and the branches thereof, and have devised methods, an apparatus, and methods of using such an apparatus, which addresses the shortcomings of previous treatments for bladder dysfunction. In particular, WO2017/066572 describes high intensity stimulation of the pudendal nerve during the filling phase to increase bladder capacity.

It is surprisingly demonstrated for the first time herein that burst stimulation of a sensory pudendal nerve improves bladder capacity when applied shortly before the onset of voiding. This means the signal does not need to be continuously applied.

The advantage of this is that the battery life of any apparatus applying the signal is prolonged, thereby making the apparatus more efficient and providing greater convenience for both the patient and clinicians. In addition, any discomfort experienced by the subject as a result of the stimulation is more limited than when stimulation is continuously applied during filling. Further, accommodation to the stimulation, leading to tachyphylaxis, will be reduced with shorter periods of stimulation.

Bladder function is comprised of two phases: a filling phase (urine storage) and a voiding phase (urine evacuation). It is therefore desirable to improve the function of either phase, preferably both phases, in a patient experiencing bladder dysfunction.

As described in the Examples, burst stimulation of the compound pudendal nerve can improve voiding efficiency, for example in female subjects. The results reported herein also indicate burst stimulation of the pudendal nerve may promote continence, for example in male subjects. Similar effects are observed as a result of burst stimulation of the sacral root of the compound pudendal nerve.

The sensory pudendal nerve burst stimulation applied to improve bladder capacity or the compound pudendal nerve stimulation applied to improve voiding efficiency can be applied individually to improve bladder function in a subject. Alternatively, both types of stimulation are applied in order to improve both bladder capacity and voiding efficiency.

Therefore, in a first aspect, the present disclosure provides an apparatus for stimulating neural activity in a pudendal nerve of a subject, the apparatus comprising, consisting of, or consisting essentially of:

at least one primary electrode configured to apply a first electrical signal to said nerve; and
a controller coupled to said primary electrode(s) and controlling the first electrical signal to be applied thereby, wherein said controller is configured to cause said at least one primary electrode to apply said first electrical signal that stimulates neural activity in the pudendal nerve to improve bladder function, wherein the first electrical signal comprises an AC waveform having a frequency in the range of from 0.1-100 Hz, for example 0.1-50 Hz or 10-100 Hz, and wherein the first electrical signal is applied in a burst pattern.

In a further aspect, the present disclosure provides a method of treating bladder dysfunction in a subject comprising, consisting of, or consisting essentially of:

i. implanting in the subject an apparatus according to the first aspect;
ii. positioning at least one primary electrode of the apparatus in signalling contact with a pudendal nerve of the subject and, when the apparatus comprises at least one secondary electrode, positioning said at least one secondary electrode of the apparatus in signalling contact with a pudendal nerve of the subject;
iii. activating the apparatus to apply an electrical signal to the pudendal nerve of the subject as caused by the controller.

In a further aspect, the present disclosure provides a method of treating bladder dysfunction in a subject comprising, consisting of, or consisting essentially of applying a first electrical signal to a pudendal nerve of the subject to stimulate activity in said pudendal nerve, wherein the first electrical signal comprises an AC waveform having a frequency in the range of from 0.1-50 Hz and wherein the first electrical signal is applied in a burst pattern.

In certain embodiments of all aspects, the first electrical signal has an amplitude in the range of from 0.05 to 10 T. In some embodiments, the first electrical signal has an amplitude in the range of from 0.1 to 10 T. In certain embodiments, the first electrical signal has an amplitude in the range of from 0.05 to 5 T, 0.3 T to 3 T, optionally the first electrical signal has an amplitude in the range of from 1 T to 3 T, optionally in the range of from 1 T to 2.2 T. In certain embodiments the first electrical signal has an amplitude of 1 T, 1.5 T or 2 T. In such embodiments “T” is the threshold amplitude which can be determined as described herein.

In certain embodiments of all aspects, the burst pattern of the first electrical signal comprises a signal burst having a duration in the range of from 20 ms to 2000 ms, optionally in the range of from 20 ms to 500 ms, optionally a duration in the range of from 20 ms to 200 ms. In certain preferred embodiments the burst pattern of the first electrical signal comprises a signal burst having a duration in the range of from 20 ms to 100 ms, optionally in the range of from 50 ms to 100 ms. In certain preferred embodiments the burst pattern of the first electrical signal comprises a signal burst having a duration of 50 ms. In certain preferred embodiments the burst pattern of the first electrical signal comprises a signal burst having a duration of 100 ms.

In certain embodiments of all aspects the burst pattern comprises a signal burst repeated at an interval of from 0.1 s to 2 s, preferably wherein the burst pattern has a signal burst repeated at an interval of from 0.125 s to 1 s. In certain embodiments the burst pattern comprises a signal burst repeated at an interval of 0.125 s, 0.2 s, or 0.5 s.

In certain embodiments of all aspects the burst pattern of the first signal comprises a signal burst having a duration of 100 ms repeated at an interval of 0.5 s, optionally wherein the burst pattern has a signal burst having a duration of 100 ms repeated at an interval of 0.5 s.

In certain embodiments of all aspects the burst pattern of the first signal comprises a signal burst having a duration of 50 ms repeated at an interval of 0.5 s, optionally wherein the burst pattern has a signal burst having a duration of 50 ms repeated at an interval of 0.5 s.

In certain embodiments of all aspects the first electrical signal is applied in a burst pattern comprising a signal burst, wherein the signal burst is repeated at a frequency in the range of from 0.5-20 Hz, optionally in the range of from 1-20 Hz, optionally at a frequency in the range of from 1-10 Hz, optionally in the range of from 2-8 Hz. In certain embodiments the first electrical signal is applied in a burst pattern comprising a signal burst, wherein the signal burst is repeated at a frequency of 2 Hz, 4.76 Hz or 8 Hz.

In certain embodiments of all aspects the first electrical signal comprises a signal burst wherein the signal burst comprises an AC waveform having a frequency in the range of from 10-100 Hz, optionally in the range of from 20-50 Hz, optionally in the range of from 30-40 Hz, optionally at a frequency of 40 Hz.

In certain embodiments of all aspects, the first electrical signal is (to be) applied to sensory fibres of a pudendal nerve, for example a sensory pudendal nerve such as the dorsal genital nerve (DGN).

It is demonstrated herein that when the first electrical signal is applied to a sensory pudendal nerve it can promote continence even when applied at the point of onset of voiding, shortly before the onset of voiding, and/or at or shortly after the onset of urine leakage.

As demonstrated in the Examples, application of the first signal to a sensory pudendal nerve can confer an increase in bladder capacity when applied shortly before the onset of voiding or at the point of voiding onset.

It is further demonstrated that application of the first signal to a sensory pudendal nerve can reduce urine output even after the onset of urine leakage, thereby reducing further leakage and promoting continence. In this context, a urine leakage is an unwanted output or voiding of urine.

Therefore in certain embodiments, the first electrical signal is (to be) applied to sensory fibres of a pudendal nerve, optionally a sensory branch of the pudendal nerve, at or shortly after onset of urine leakage.

It is further demonstrated in the Examples that stimulation of the compound pudendal nerve or sacral root during the voiding phase can improve voiding efficiency, thereby reducing unwanted urine retention.

Therefore, in certain alternative embodiments, the first signal is (to be) applied to a compound pudendal nerve or the sacral root. In certain such embodiments, the effect of applying the signal is to increase voiding efficiency, for example in certain embodiments where the subject is female. In certain embodiments, the effect of applying the signal is to improve continence, for example in certain embodiments where the subject is male.

In certain such embodiment the first electrical signal is (to be) applied during a voiding phase of the micturition cycle.

In certain embodiments of all aspects, a second electrical signal is (to be) applied. In such embodiments, the first electrical signal is applied to sensory fibres of a pudendal nerve, for example a sensory branch of the pudendal nerve, so as to increase bladder capacity. In certain such embodiments the second electrical signal is applied to a compound pudendal nerve or sacral root so as to increase voiding efficiency, for example in certain embodiments where the subject is female. In certain embodiments, the effect of applying the second electrical signal is to increase continence, for example in certain embodiments where the subject is male.

In an alternative such embodiment, the second signal is applied to a pudendal motor nerve so as to increase voiding efficiency. Stimulation of a pudendal motor nerve to promote voiding efficiency is demonstrated in the Examples and also described in WO2017/066572.

In those embodiments where a second electrical signal is (to be) applied, the parameters and bursting pattern of the second signal may be independently selected from the same options set out herein in relation to the first signal.

Apparatuses and methods according to the invention have the further advantage that they can be used in conjunction with pharmaceutical therapies for bladder dysfunction to reduce symptoms.

Therefore, in a further aspect the invention provides a pharmaceutical composition comprising a compound for treating bladder dysfunction, for use in a method of treating bladder dysfunction in a subject, wherein the method is a method according to the invention, the method further comprising the step of administering an effective amount of the pharmaceutical composition to the subject.

In a further aspect, the invention provides a pharmaceutical composition comprising a compound for treating bladder dysfunction, for use in treating bladder dysfunction in a subject, the subject having an apparatus according to the invention implanted.

In certain such embodiments, the compound for treating bladder dysfunction is an antimuscarinic compound or a β-adrenergic receptor agonist, optionally a β3-adrenergic receptor agonist. In certain embodiments, the antimuscarinic compound is selected from darifenacin, hyoscyamine, oxybutynin, tolterodine, solifenacin, trospium, or fesoterodine. In certain embodiments, the β3-adrenergic receptor agonist is mirabegron.

In a further aspect, the invention provides a neuromodulation system comprising a plurality of apparatuses according to the invention. In certain embodiments, each apparatus is arranged to communicate with at least one other apparatus in the system, optionally all apparatuses in the system. In certain embodiments, the system further comprises a processor arranged to communicate with the apparatuses of the system.

In a preferred embodiment of all aspects of the invention, the subject to be treated or for which the apparatus is to be used is a human subject.

In certain embodiments the subject is a male subject. In certain embodiments the subject is a female subject.

In all aspects, unless specified otherwise, “pudendal nerve” refers to the pudendal nerve and its branches. In certain embodiments, the first and/or second electronic signal is (to be) applied to a sensory branch of the pudendal nerve (also referred to herein as “a sensory pudendal nerve”). In certain such embodiments, the first and/or second electronic signal is (to be) applied to a dorsal genital nerve (DGN). In certain embodiments, the first and/or second electronic signal is (to be) applied to the compound pudendal nerve or to the sacral root.

Another aspect of the present disclosure provides all that is disclosed and illustrated herein.

### BRIEF DESCRIPTION OF FIGURES

FIG. 1 Schematic drawings showing how apparatuses, devices and methods according to the invention can be put into effect.

FIG. 2 Effect of bursting stimulation applied to the pudendal sensory branch during voiding. (A) Example cystometrograms from a female cat. The upper trace shows control bladder pressure and voiding in no-stimulation conditions. In the lower trace, continuous pudendal sensory branch stimulation during the filling phase increased bladder capacity, and application of bursting stimulation to the same site at or immediately prior to void onset inhibited ongoing bladder contractions and stopped urine output. (B) Summary of the effect of pudendal sensory branch bursting stimulation on voiding efficiency in cats (male n=4; female, n=2). Except for one male cat, pudendal sensory branch bursting decreased voiding efficiency relative to controls.

FIG. 3 Bursting stimulation of the sensory pudendal nerve in the female rat. (A) Example cystometrograms showing a no-stimulation trial and a stimulation trial that included sensory pudendal bursting at a 4.76 Hz train rate. The onset of stimulation occurred at a volume that was expected to be just before the bladder contraction in the previous control trial (top trace). Bursting stimulation inhibited bladder contractions. (B) Effect of several different bursting rates was compared (2 Hz, 4.76 Hz, and 8 Hz). Sensory bursting increased bladder capacity by at least 40% for all bursting rates.

FIG. 4 Bursting stimulation on the compound pudendal nerve during voiding. (A+B) Example cystometrograms from two experiments, with a no-stimulation trial (left), compound pudendal nerve bursting (middle), and pudendal motor branch bursting (right). (C) Voiding efficiency values from three experiments, comparing the effect of no-stimulation during voiding against bursting stimulation on the compound pudendal nerve or pudendal motor branch intervention sites. (D and E) Voiding efficiency values comparing the effect of no-stimulation against bursting stimulation on the compound pudendal nerve or sacral root (at low or high amplitudes) of female (D) and male (E) cats.

### DETAILED DESCRIPTION OF THE INVENTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

The terms as used herein are given their conventional definition in the art as understood by the skilled person, unless otherwise defined below. In the case of any inconsistency or doubt, the definition as provided herein should take precedence.

Articles “a” and “an” are used herein to refer to one or to more than one (i.e. at least one) of the grammatical object of the article. By way of example, “an element” means at least one element and can include more than one element.

The use herein of the terms “including,” “comprising,” or “having,” and variations thereof, is meant to encompass the elements listed thereafter and equivalents thereof as well as additional elements. Embodiments recited as “including”, “comprising”, or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a frequency range is stated as 1 Hz to 50 Hz, it is intended that values such as 2 Hz to 40 Hz, 10 Hz to 30 Hz, or 1 Hz to 3 Hz, etc., are expressly enumerated in this specification. These are only examples of what is specifically intended, and all possible combinations of numerical values between and including the lowest value and the highest value enumerated are to be considered to be expressly stated in this disclosure.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, rat, horse, cow, chickens, amphibians, reptiles, and the like.

As used herein, “electrode” is taken to mean any element capable of applying an electrical signal to the nerve.

As used herein, “stimulation of neural activity” may be an increase in the total signalling activity of the whole nerve, or that the total signalling activity of a subset of nerve fibres of the nerve is increased, compared to baseline neural activity in that part of the nerve. A “selective increase in neural activity”, for example in the sensory fibres, causes a preferential increase in neural activity in the sensory fibres compared to any increase in neural signalling in the motor nerve fibres of the pudendal nerve.

“Phase-specific” or “state-dependent” stimulation are taken to mean that a different stimulation is applied depending on the ongoing and/or desired phase of the normal bladder activity cycle. The bladder activity cycle or micturition cycle is characterised by a filling phase (also referred to as a storage phase), followed by a triggering of the micturition, followed by a voiding phase (also referred to as the micturition phase). A normal bladder activity cycle is a bladder activity cycle characteristic of a healthy individual.

Application of an electrical signal in a “burst pattern” refers to application of the signal in a series of bursts. That is, the signal is applied for a burst—that is, a duration of time—followed by an interval in which no signal is applied. The interval in which no signal is applied is also referred to as the train rate. The interval is then followed by another burst, followed by another interval. The burst pattern is the combination of the burst for which the signal is applied followed by the interval during which no signal is applied.

The “ongoing phase” of bladder activity is the phase of the bladder activity cycle occurring at a particular given time. That a subject is in a given phase of the cycle can be indicated by a physiological parameter relevant to bladder activity, for example bladder pressure. For example, that a subject is in the filling phase may be indicated by increasing bladder pressure, or a sustained bladder pressure indicating that the bladder is at least partially filled. Triggering of micturition may be indicated by a sharp increase in bladder pressure. Other physiological parameters relevant to bladder activity include nerve activity in the pudendal nerve, nerve activity in the hypogastric nerve, nerve activity in the pelvic nerve, muscle activity in the bladder detrusor muscle, muscle activity in the internal urethral sphincter, muscle activity in the external urethral sphincter (EUS), muscle activity in the external anal sphincter (EAS).

The “desired phase” of the bladder activity cycle is the phase of the bladder activity cycle of which the subject is desirous. The desired phase may depend on the behaviour of the subject, for example whether they are sleeping, at exercise, at work, etc. Similarly, the desired phase may depend on perceived levels of urinary comfort. For example, the subject may perceive discomfort due to the sensation of having a full bladder, and therefore be desirous of triggering micturition.

It will be appreciated that phase-specific stimulation can take into account both ongoing and desirous phases of the bladder activity cycle. For example, a first stimulating signal may be applied (e.g. to increase bladder capacity) during a filling phase indicated by increasing bladder pressure, and a second stimulating signal may be applied when the subject is desirous of beginning micturition (e.g. to trigger micturition), or during a voiding phase as indicated by a change in muscle activity in the EUS (e.g. to increase voiding efficiency).

As used herein “a pudendal nerve” refers to the compound pudendal nerve and its associated branches, for example the dorsal genital nerve of the penis/clitoris (DGN).

As used herein, a “healthy individual” or “healthy subject” is an individual not exhibiting any disruption or perturbation of normal bladder activity.

As used herein, “bladder dysfunction” is taken to mean that the patient or subject is exhibiting disruption of bladder function compared to a healthy individual. Bladder dysfunction may be characterised by symptoms such as nocturia, increased urinary retention, increased incontinence, increased urgency of urination or increased frequency of urination compared to a healthy individual. Symptoms may also include sensation of incomplete emptying, straining to void, and recurrent urinary tract infections. Bladder dysfunction includes conditions such as overactive bladder (OAB), neurogenic bladder, stress incontinence, underactive bladder (UAB), and urinary retention.

Treatment of bladder dysfunction, as used herein may be characterised by any one or more of a reduction in number of incontinence episodes, a decrease in urgency of urination, a decrease in frequency of urination, an increase bladder capacity, an increase in bladder voiding efficiency, a decrease in urine leakage, a decrease in urinary retention, a change in external urethral sphincter (EUS) activity towards that of a healthy individual, and/or a change in the pattern of action potentials or activity of the pudendal nerve towards that of a healthy individual.

The skilled person will appreciate that the baseline for any neural activity or physiological parameter in an individual need not be a fixed or specific value, but rather can fluctuate within a normal range or may be an average value with associated error and confidence intervals. Suitable methods for determining baseline values would be well known to the skilled person. Baseline neural activity occurs prior to an application of a signal.

As used herein, a measurable physiological parameter is detected in a subject when the value for that parameter exhibited by the subject at the time of detection is determined. A detector is any element able to make such a determination.

A “predefined threshold value” for a physiological parameter is the value for that parameter where that value or beyond must be exhibited by a subject or subject before the intervention is applied. For any given parameter, the threshold value may be defined as a value indicative of a particular physiological state. Examples of such predefined threshold values include: bladder pressure indicative of bladder at or near capacity, EUS activity patterns indicative of imminent onset of bladder voiding. Such a threshold value for a given physiological parameter is exceeded if the value exhibited by the subject is beyond the threshold value—that is, the exhibited value is a greater departure from the normal or healthy value for that parameter than the predefined threshold value.

The measurable physiological parameter may comprise an action potential or pattern of action potentials in one or more nerves of the subject, wherein the action potential or pattern of action potentials is associated with bladder dysfunction. Suitable nerves in which to detect an action potential or pattern of action potentials include a pudendal nerve, a pelvic nerve and/or a hypogastric nerve. In a particular embodiment, the measurable physiological parameter comprises the pattern of action potentials in the pudendal nerve.

The measurable physiological parameter may be muscle electromyographic activity, wherein the electromyographic activity is indicative of the level of activity in the muscle. Such activity could typically be measured from the bladder detrusor muscle, the internal urethral sphincter, the external urethral sphincter, and the external anal sphincter.

As used herein, “implanted” is taken to mean positioned within the subject's body. Partial implantation means that only part of the apparatus is implanted—i.e. only part of the apparatus is positioned within the subject's body, with other elements of the apparatus external to the subject's body. Wholly implanted means that the entire apparatus is positioned within the subject's body. For the avoidance of doubt, the apparatus being “wholly implanted” does not preclude additional elements, independent of the apparatus but in practice useful for its functioning (for example, a remote wireless charging unit or a remote wireless manual override unit), being independently formed and external to the subject's body.

In WO2017066572 it was reported that stimulating the motor component of the pudendal nerve using an electrical signal in a burst pattern resulted in improved voiding efficiency. As shown for the first time in the Examples below, burst stimulation of the sensory pudendal nerve is able to promote continence (e.g. bladder capacity) and healthy bladder activity in a subject, for example a subject wishing to have greater control over micturition.

Thus, in accordance with a first aspect of the invention, there is provided an apparatus for stimulating neural activity in a pudendal nerve of a subject, the apparatus comprising: at least one primary electrode configured to apply a first electrical signal to said nerve; and a controller coupled to said primary electrode(s) and controlling the first electrical signal to be applied thereby, wherein said controller is configured to cause said at least one primary electrode to apply said first electrical signal that stimulates neural activity in the pudendal nerve to improve bladder function, wherein the first electrical signal comprises an AC waveform having a frequency in the range of from 0.1-100 Hz, optionally 0.1-50 Hz, and wherein the first electrical signal is applied in a burst pattern.

In certain embodiments the first electrical signal has an amplitude in the range 0.05 to 10 T. In other embodiments the first electrical signal has an amplitude in the range 0.1 to 10 T.

“T” is a measure of relative stimulation intensity. Relative stimulation intensity can be expressed as multiples (0.1, 0.8, 1, 2, 5, etc.) of “T”. “T” represents the threshold stimulation intensity to evoke a motor response. For example, “1 T” is defined as the threshold stimulation intensity required to evoke a motor response—in particular, as used herein in relation to sensory pudendal stimulation “T” may be defined as the threshold amplitude required to evoke a reflex electromyogram (EMG) response in the external urethral sphincter (EUS) when the electrical signal is applied to the pudendal nerve.

Other motor reflexes can be used to determine T—for example, the reflex response in the external anal sphincter (EAS) following pudendal nerve stimulation. Values for T determined using the EAS are equivalent to those determined using the EUS.

Determining “T” as described herein provides a calibration baseline able to be transferred between individuals and/or species. T thus provides a useful measure for amplitude normalization between individuals and/or species. For example, T may be determined as follows: a low frequency electrical signal (e.g., 1 Hz) is applied and the intensity of stimulation is increased (either by increasing the voltage or the current of the signal, preferably the current) until the pudendal nerve stimulation produces a reflex EMG response in the EUS. This stimulation intensity is designated T. The absolute threshold stimulation intensity may vary across individuals and/or species due to inherent variation, positioning and type of the electrode, etc., and therefore subsequent experimental or therapeutic intensities are designated as multiples of T to provide equivalent relative stimulation intensities.

The desired stimulation intensity (i.e. the desired multiple of threshold intensity “T”) can be achieved through controlled variation of the current or voltage of the signal, preferably the current.

In some embodiments, the first electrical signal has an amplitude in the range from 0.05 T to 5.0 T. In certain embodiments, the first signal has an amplitude in the range from 0.3 T to 3 T. In some embodiments, the first electrical signal has an amplitude of 0.05, 0.1, 0.2, 0.3, 0.4, 0.5 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 T.

In certain embodiments, the first electrical signal has an amplitude value in the range of from 0.5 T to 3 T, preferably in the range of from 1 T to 3 T. In certain embodiments, the first electrical signal has an amplitude value in the range of from 1 T to 2.2 T. In certain embodiments, the first electrical signal has an amplitude value of at least 1 T. In certain embodiments, the first electrical signal has an amplitude value of at least 1.2 T, optionally at least 1.3 T, optionally at least 1.4 T, optionally at least 1.5 T, optionally at least 1.6 T, optionally at least 1.7 T, optionally at least 1.8 T, optionally at least 1.9 T, optionally at least 2 T.

In some embodiments, the electrical signal has an amplitude of 1 T, 1.5 T, 1.8 T, 2 T, 2.3 T, 2.5 T or 3 T. In certain preferred embodiments the first electrical signal has an amplitude in the range of from 1.8 T to 3 T, preferably 1.8 T to 2.3 T. In certain preferred embodiments the first electrical signal has an amplitude in the range of from 2 T to 3 T.

In other embodiments, the first electrical signal has an amplitude of 1 T. In other embodiments, the first electrical signal has an amplitude of 1.5 T. In other embodiments the first electrical signal has an amplitude of 2 T. In other embodiments the first electrical signal has an amplitude of 3 T.

In other embodiments, the first electrical signal has an amplitude in the range of from 0.1-20 mA, optionally 0.1-10 mA, optionally 0.1-5 mA, optionally 0.1-1 mA, optionally 100-500 μA, optionally 100-400 μA. In certain embodiments, the first electrical signal has an amplitude of 100 μA, 200 μA, 300 μA or 400 μA.

In certain embodiments, the burst pattern of the first electrical signal comprises a signal burst having a duration in the range of from 20 ms to 2000 ms, optionally in the range of from 20 ms to 500 ms, optionally a duration in the range of from 20 ms to 200 ms. In certain preferred embodiments the burst pattern of the first electrical signal comprises a signal burst having a duration in the range of from 20 ms to 100 ms, optionally in the range of from 50 ms to 100 ms. In certain preferred embodiments the burst pattern of the first electrical signal comprises a signal burst having a duration of 50 ms. In certain preferred embodiments the burst pattern of the first electrical signal comprises a signal burst having a duration of 100 ms.

In certain embodiments, the burst pattern of the first electrical signal comprises a signal burst repeated at an interval of from 0.1 to 2 s, optionally 0.125 s to 2 s. In certain embodiments, the burst pattern comprises a signal burst repeated at an interval of from 0.125 s to 1 s. In certain embodiments, the burst pattern comprises a signal burst repeated at an interval of from 0.125 s to 0.5 s. In certain embodiments, the burst pattern comprises a signal burst repeated at an interval of 0.125 s. In certain embodiments, the burst pattern comprises a signal burst repeated at an interval 0.2 s. In certain embodiments, the burst pattern comprises a signal burst repeated at an interval of 0.5 s.

In certain embodiments, the burst pattern of the first electrical signal comprises a signal burst having a duration from 20 ms to 2000 ms repeated at an interval of from 0.1 s to 1 s. In certain embodiments, the burst pattern comprises a signal burst having a duration from 20 ms to 500 ms repeated at an interval of from 0.125 s to 0.5 s.

In certain embodiments the burst pattern of the first electrical signal comprises a signal burst having a duration of 50 ms repeated at an interval of 0.5 s, optionally wherein the burst pattern has a signal burst having a duration of 50 ms repeated at an interval of 0.5 s.

In certain embodiments the burst pattern of the first electrical signal comprises a signal burst having a duration of 50 ms repeated at an interval of 0.2 s, optionally wherein the burst pattern has a signal burst having a duration of 50 ms repeated at an interval of 0.2 s.

In certain embodiments the burst pattern of the first electrical signal comprises a signal burst having a duration of 50 ms repeated at an interval of 0.125 s, optionally wherein the burst pattern has a signal burst having a duration of 50 ms repeated at an interval of 0.125 s.

In certain embodiments the burst pattern of the first electrical signal comprises a signal burst having a duration of 100 ms repeated at an interval of 0.5 s, optionally wherein the burst pattern has a signal burst having a duration of 100 ms repeated at an interval of 0.5 s.

In certain embodiments the burst pattern of the first electrical signal comprises a signal burst having a duration of 100 ms repeated at an interval of 0.2 s, optionally wherein the burst pattern has a signal burst having a duration of 100 ms repeated at an interval of 0.2 s.

In certain embodiments the burst pattern of the first electrical signal comprises a signal burst having a duration of 100 ms repeated at an interval of 0.125 s, optionally wherein the burst pattern has a signal burst having a duration of 100 ms repeated at an interval of 0.125 s.

In some embodiments, the first electrical signal applied in a burst pattern comprises a signal burst repeated at a frequency in the range of from 0.5 to 20 Hz. For example, a burst pattern comprising a signal burst at 40 Hz repeated at a frequency of 2 Hz would repeat the signal burst at 0.5 s intervals (see diagram below).

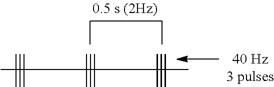

In some embodiments, the first electrical signal applied in a burst pattern comprises a signal burst repeated at a frequency in the range of 1 to 20 Hz. In some embodiments, the first electrical signal applied in a burst pattern comprises a signal burst repeated at a frequency in the range of 2 to 10 Hz, preferably in the range of from 2 Hz to 8 Hz.

In one embodiment, the first electrical signal comprises a signal burst repeated at a frequency comprising 10 Hz, optionally wherein the first electrical signal comprises an AC waveform repeated at a frequency of 10 Hz. In another embodiment, the first electrical signal is repeated at a frequency comprising 2 Hz, optionally wherein the first electrical signal is repeated at a frequency of 2 Hz. In another embodiment, the first electrical signal is repeated at a frequency comprising 4.76 Hz, optionally wherein the first electric signal is repeated at a frequency of 4.76 Hz. In another embodiment, the first electrical signal is repeated at a frequency comprising 8 Hz, optionally wherein the first electric signal is repeated at a frequency of 8 Hz.

In certain embodiments, the first electrical signal comprises a signal burst wherein the signal burst comprises an AC waveform having a frequency in the range of from 10-100 Hz. In certain embodiments, the first electrical signal comprises a signal burst wherein the signal burst comprises an AC waveform having a frequency in the range of from 0.1-50 Hz. In certain embodiments, the first electrical signal comprises a signal burst wherein the signal burst comprises an AC waveform having a frequency in the range of from 20-50 Hz. In certain embodiments, the signal burst comprises an AC waveform having a frequency in the range of from 30-50 Hz, optionally in the range of from 30-40 Hz. In certain such embodiments, the signal burst has a frequency of 40 Hz.

In some embodiments, the burst pattern consists of from 1 to 10 pulses per signal burst. In some embodiments, the burst pattern consists of from 1 to 5 pulses per signal burst. In some embodiments, the burst pattern consists of 3 or 4 pulses per signal burst. In such embodiments, the duration of the signal burst is thus determined by the frequency of the AC waveform.

It will be appreciated by the skilled person that an electronic signal “comprising” an indicated frequency may have other frequency components as part of the signal. In certain preferred embodiments of all aspects where a signal comprises an indicated frequency, the indicated frequency is the dominant frequency component of the signal.

In certain embodiments, the AC waveform is a biphasic waveform, optionally a charge-balanced biphasic waveform. In certain such embodiments, the waveform may be symmetrical or asymmetrical. In certain such embodiments, each phase of the biphasic waveform has a phase duration from 0.005 ms to 2 ms, optionally 0.01 to 1 ms, optionally 0.05 to 0.5 ms, optionally 0.05 to 0.2 ms, optionally 0.1 ms. In certain embodiments, each phase of a biphasic waveform is of equal duration. In certain alternative embodiments, each phase is of a different duration.

The AC waveform may be selected from pulsatile, sinusoidal, triangular, rectangular, square or a complex waveform.

In certain embodiments, the apparatus may comprise two or more primary electrodes, where each primary electrode is configured to apply the first electronic signal. In certain such embodiments, the apparatus comprises two primary electrodes suitable for bilateral positioning.

It is demonstrated herein for the first time that stimulating sensory fibres of the pudendal nerve using an electrical signal in a burst pattern is able to increase bladder capacity. As shown in the Examples, bursting stimulation of the sensory pudendal nerve (“pudendal sensory bursting”) initiated at or just prior to the onset of voiding delays micturition and promotes bladder capacity.

It is further demonstrated herein that stimulating sensory fibres of the pudendal nerve using an electrical signal in a burst pattern is able to reduce urine leakage. As shown in the Examples, bursting stimulation of the sensory pudendal nerve initiated at or just after to the onset of urine leakage can reduce the volume and/or duration of urine leakage.

For a subject experiencing a sensation of urgency, pudendal sensory bursting is therefore able to provide an additional time window before voiding onset. This allows the subject more control as well as time to locate a bathroom or restroom.

Therefore, in certain embodiments, the first electrical signal to be applied by the apparatus stimulates neural activity in sensory fibres of the pudendal nerve, optionally selectively stimulates neural activity in sensory fibres of the pudendal nerve, so as to produce an increase in bladder capacity.

In certain preferred embodiments, the pudendal nerve to be stimulated by the first electrical signal is a sensory pudendal nerve of the subject. In certain embodiments, the sensory pudendal nerve is the dorsal nerve of the penis/clitoris (DNP, also known as the dorsal genital nerve (DGN)).

In such embodiments where the first signal promotes bladder capacity, the controller is configured to apply the first signal during the filling phase.

The sensory bursting is particularly advantageous as it is able to delay voiding when applied towards the end of the filling phase, at or shortly prior to voiding onset. Applying the first signal at this point means there is no ongoing signal being applied during filling, which otherwise might cause discomfort to the subject when in use, and which would put greater demand on power supply and storage. This is in contrast to WO2017066572, where continuous high amplitude stimulation of the sensory pudendal nerve during filling was required to affect bladder capacity.

Therefore in certain preferred embodiments, the controller is configured to begin to apply the first electrical signal at or shortly prior to onset of voiding, for example immediately prior to voiding onset. In such embodiments, no signal is otherwise applied during the filling phase. Voiding onset can be determined by monitoring one or more indicators of imminent voiding, such as bladder pressure approaching a threshold indicative of imminent voiding, or EUS activity increasing to a level indicative of imminent voiding. Application of the first signal in this manner promotes bladder capacity.

A further advantage of the pudendal sensory bursting stimulation is that, when applied at or shortly after the onset of urine leakage, it results in improved continence. As demonstrated in the Examples, application of pudendal sensory bursting (that is, application of the first signal to a sensory pudendal nerve (e.g. DGN)) once leakage has commenced can reduce urine output, thereby promoting continence—that is, reducing unwanted urine output or leakage even once a leak has begun.

Therefore, in certain embodiments, the controller is configured to apply the first electrical signal at or shortly after leakage onset. In this context, urine leakage may be indicated or detected by changes in bladder pressure, bladder detrusor activity and/or EUS activity characteristic of leakage. In such embodiments, application of the first electrical signal promotes continence, for example by reducing unwanted urine output.

Once voiding has begun, for some subjects it is desirable for voiding efficiency to be increased—for example, subjects who otherwise experience a sensation of partial bladder emptying (e.g. patients suffering from underactive bladder (UAB) would benefit from increased voiding efficiency.

As demonstrated in the Examples, bursting stimulation of the compound pudendal nerve results in improved (e.g. increased) voiding efficiency when the bursting stimulation is applied during the voiding phase. This contrasts with WO2017066572, which describes bursting stimulation applied to the motor branch of the pudendal nerve. In contrast to the more-distal motor branch of the pudendal nerve, the compound pudendal nerve is a mixed nerve including both sensory and motor fibres. (The effect of bursting stimulation of the motor branch of the pudendal nerve described in WO2017066572 is confirmed by the data provided in the present Examples.)

Therefore, in certain embodiments the first electrical signal to be applied by the apparatus stimulates neural activity in the compound pudendal nerve, so as to improve bladder function, for example so as to increase voiding efficiency or so as to improve continence. In such embodiments, the controller is configured to apply the first electrical signal during the voiding phase. In certain embodiments the first electrical signal to be applied by the apparatus stimulates neural activity in the compound pudendal nerve, so as to increase voiding efficiency. In certain such embodiments preferably the subject is female. In certain embodiments the first electrical signal to be applied by the apparatus stimulates neural activity in the compound pudendal nerve, so as to improve continence. In such embodiments, preferably the subject is male.

Bursting stimulation of the sacral root (S1, S2, S3, or S4) is expected to have the same effect as that reported below for the compound pudendal nerve. As demonstrated in the Examples, bursting stimulation of the sacral root can provide improved bladder function, for example increased voiding efficiency or improved continence. Preferably the sacral root (to be) stimulated is the S1, S2, S3 or S4 sacral root, or a combination thereof. Preferably the sacral root (to be) stimulated is the S2, S3, or S4 sacral root, or a combination thereof. Preferably the sacral root (to be) stimulated is the S2 sacral root. Preferably the sacral root (to be) stimulated is the S3 sacral root. Preferably the sacral root (to be) stimulated is the S4 sacral root.

Therefore, in certain embodiments the first electrical signal to be applied by the apparatus stimulates neural activity in the sacral root, so as to increase voiding efficiency. In certain such embodiments, preferably the subject is female. In certain embodiments the first electrical signal to be applied by the apparatus stimulates neural activity in the sacral root, so as to improve continence. In certain such embodiments, preferably the subject is male.

The amplitude “T” of the electrical signal (to be) applied to the sacral root can be selected according to the embodiments provided elsewhere herein. In certain embodiments wherein the electrical signal is (to be) applied by the apparatus to the sacral root, the electrical signal has an amplitude of at least 1 T, optionally an amplitude of at least 1.5 T, optionally at least 2 T.

It is a further advantage that the improvement in bladder capacity and the increase in voiding efficiency can be combined by applying a first and a second electrical signal in a phase-specific manner—the first during filling phase (preferably towards the end of the filling phase), and the second during voiding phase. In certain such embodiments, the first and second signals are applied by the same electrode.

Therefore, in another embodiment, the apparatus further comprises at least one secondary electrode configured to apply a second electrical signal to a compound pudendal nerve, a sacral root, or a pudendal motor nerve; and

a controller coupled to said secondary electrode(s) and controlling the second electrical signal to be applied thereby, wherein said controller is configured to cause said at least one secondary electrode to apply said second electrical signal that stimulates neural activity in the compound pudendal nerve, the sacral root, or the pudendal motor nerve to improve voiding efficiency, wherein the second electrical signal comprises an AC waveform having a frequency in the range of from 0.1-100 Hz, optionally 0.1-50 Hz, and wherein the second electrical signal is applied in a burst pattern.

In such embodiments, the first electrical signal is applied to a sensory pudendal nerve so as to increase bladder capacity in accordance with such embodiments provided herein, and the second electrical signal is applied to a compound pudendal nerve, sacral root or pudendal motor nerve so as to increase voiding efficiency in accordance with such embodiments provided herein.

In preferred such embodiments the controller is configured to apply the first electrical signal shortly prior to onset of voiding, for example immediately prior to voiding onset, and the controller is configured to apply the second signal during the voiding phase. In preferred such embodiments, no signal is otherwise applied during the filling phase.

In such embodiments, the first electrical signal is to be applied to a sensory pudendal nerve, for example the DGN, and the second electrical signal is to be applied to the compound pudendal nerve or sacral root. In an alternative embodiment, the first electrical signal is to be applied to a sensory pudendal nerve, for example the DGN, and the second electrical signal is to be applied to a pudendal motor nerve.

In those embodiments where a second signal is to be applied, the embodiments and preferred embodiments of the signal parameters (e.g. amplitude, frequency, burst pattern) provided above in relation to the first electrical signal apply equally and independently to the second electrical signal.

In certain embodiments, the first electrical signal and second electrical signal have an amplitude that is the same.

In certain embodiments, the first electrical signal and second electrical signal have an amplitude that is different.

In certain embodiments, the first electrical signal and second electrical signal have a frequency that is the same.

In certain embodiments, the first electrical signal and second electrical signal have a frequency that is different.

In certain embodiments, the first electrical signal and second electrical signal have a burst pattern that is the same.

In certain embodiments, the first electrical signal and second electrical signal have a burst pattern that is different.

In certain embodiments, the apparatus may comprise two or more secondary electrodes, where each secondary electrode is configured to apply the second electronic signal. In certain such embodiments, the apparatus comprises two secondary electrodes suitable for bilateral positioning.

The one or more electrodes of the apparatus (whether that be the primary and/or secondary electrode(s)) may be any suitable electrodes for application of the signal, for example cuff electrodes, hook electrodes, percutaneous lead electrodes, transcutaneous lead electrodes or similar. Preferably the electrodes are cuff electrodes.

In certain embodiments, the apparatus further comprises a detector to detect one or more physiological parameters in the subject. Such a detector may be configured to detect one physiological parameter or a plurality of physiological parameters. The detected physiological parameter(s) are selected from nerve activity in the pudendal nerve, nerve activity in the hypogastric nerve, nerve activity in the pelvic nerve, muscle activity in the bladder detrusor muscle, muscle activity in the internal urethral sphincter, muscle activity in the external urethral sphincter, muscle activity in the external anal sphincter, and bladder pressure.

In such embodiments, the controller is coupled to the detector configured to detect one or more physiological parameters and causes the controller to cause the first electrical signal to be applied when the physiological parameter is detected to exhibit a first predefined characteristic, for example to be meeting or exceeding a first predefined threshold value, for example when the detected value indicates that the subject is in the filling phase of the micturition cycle. In certain preferred embodiments, the controller causes the first electronic signal to be applied when the detector detects that onset of a bladder voiding phase will shortly begin. For example the detector may detect that bladder voiding phase will shortly begin due to bladder pressure reaching a threshold value which has been determined to be indicative of imminent voiding for that subject. By way of further example, the detector may detect that bladder voiding phase will shortly begin by detecting that the pattern of EUS activity exhibited by the subject is indicative of imminent voiding for that subject.

Where a second electronic signal is to be applied, the detector may cause the controller to cause the second electrical signal to be applied when a physiological parameter is detected to exhibit a second predefined characteristic, for example to be meeting or exceeding a second predefined threshold value, for example when the detected value indicates that the subject is in the voiding phase of the micturition cycle. By way of example, the detector may detect bladder detrusor muscle activity.

It will be appreciated that any two or more of the indicated physiological parameters may be detected in parallel or consecutively. For example, in certain embodiments, the controller is coupled to a detector or detectors configured to detect the activity of the EUS at the same time as the bladder pressure in the subject.

By way of further example, the detector or detectors may detect physiological parameters indicative of behaviour in which storage phase is appropriate (e.g. sleeping or following urination, where it is desirous to promote storage). In response to such data being detected by the detector(s), the controller causes a signal to be applied that produces a physiological response appropriate for improved storage, for example increased bladder capacity when a sleep-state is detected.

In addition, or as an alternative to a detector, the apparatus may comprise an input element. In such embodiments, the input element allows the subject to enter data regarding their behaviour and/or desires. For example, the input element may allow the subject to enter that they desire to delay bladder voiding (i.e. intend to delay urinating), or that they have sensed a urinary leak. In such embodiments, the controller is configured to cause a signal to be applied that produces a physiological response appropriate to the data input—for example, in the case of the intention to delay urination being indicated, the signal may increase bladder capacity. By way of further example, the input may allow the subject to enter that they wish to begin urinating, in which case the controller may cause a signal promoting bladder capacity to stop being applied, and/or may apply a signal which promotes voiding efficiency.

By way of further example, the input element may also allow the subject to enter data indicative of behaviour in which storage phase is appropriate (e.g. sleeping or following urination, where it is desirous to promote storage). In response to such data being entered via the input element, the controller causes a signal to be applied that produces a physiological response appropriate for improved storage, for example increased bladder capacity.

The input element may be connected directly to the controller, or be in wireless communication as a remote component, for example a component carried by the subject.

Such arrangements and configurations are discussed in further detail below.

In certain embodiments, the apparatus further comprises one or more power supply elements, for example a battery, and/or one or more communication elements.

In certain embodiments, the apparatus is suitable for at least partial implantation into the subject, optionally full implantation into the subject.

In a second aspect, the present disclosure provides a method of treating bladder dysfunction in a subject comprising:

i. implanting in the subject an apparatus according to the invention;
ii. positioning at least one primary electrode of the apparatus in signalling contact with a pudendal nerve of the subject and, when the apparatus comprises at least one secondary electrode, positioning said at least one secondary electrode of the apparatus in signalling contact with a pudendal nerve of the subject;
iii. activating the apparatus to apply an electrical signal to the pudendal nerve of the subject as caused by the controller. The apparatus is activated when the apparatus is in an operating state such that the signal will be applied as determined by the controller.

In a second aspect, the present disclosure provides a method of treating bladder dysfunction in a subject comprising:

i. implanting in the subject an apparatus according to the invention;
ii. positioning at least one primary electrode of the apparatus in signalling contact with sensory fibres of a pudendal nerve of the subject, optionally a sensory branch of the pudendal nerve of the subject, and, when the apparatus comprises at least one secondary electrode, positioning said at least one secondary electrode of the apparatus in signalling contact with a compound pudendal nerve or sacral root or pudendal motor nerve of the subject;
iii. activating the apparatus to apply an electrical signal to the sensory fibres of the subject, optionally a sensory branch of the pudendal branch of the subject, and/or the compound pudendal nerve or sacral root or pudendal motor nerve of the subject as caused by the controller. The apparatus is activated when the apparatus is in an operating state such that the signal will be applied as determined by the controller.

In certain embodiments, the primary electrode is positioned in signalling contact with a sensory branch of the pudendal nerve of the subject. In certain embodiments, the sensory pudendal nerve is the dorsal nerve of the penis/clitoris (DNP, also known as the dorsal genital nerve (DGN)).

In certain embodiments, the method comprises implanting an apparatus according to the invention having at least two primary electrodes, and optionally at least two secondary electrodes, and positioning the electrodes bilaterally—that is, one primary electrode in signalling contact with a left pudendal nerve, and one primary electrode in signalling contact with a right pudendal nerve.

In certain embodiments, the method is a method for treating overactive bladder, neurogenic bladder, mixed urge and stress incontinence, under active bladder (UAB), urinary retention, or detrusor hyperactivity with impaired contractility (DHIC).

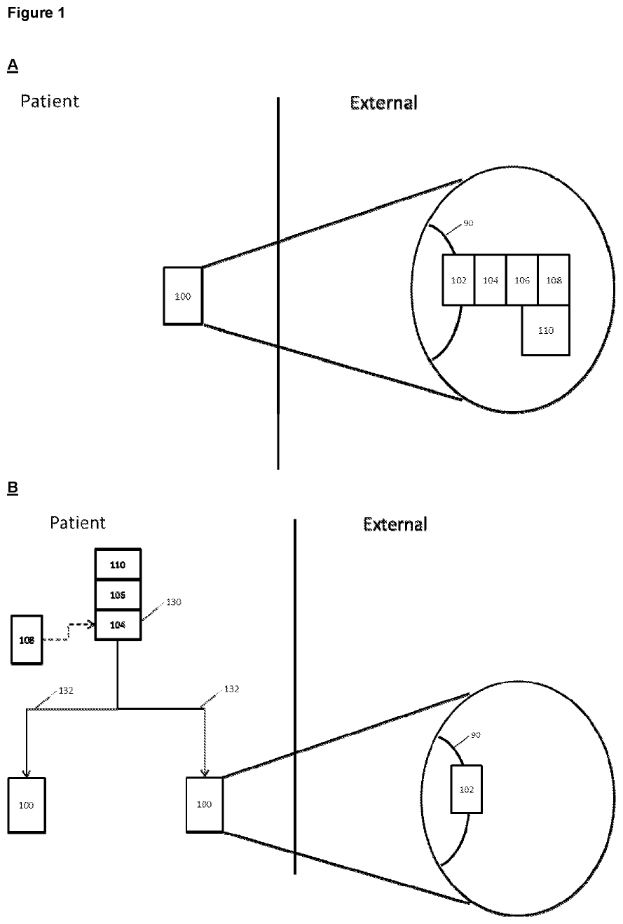

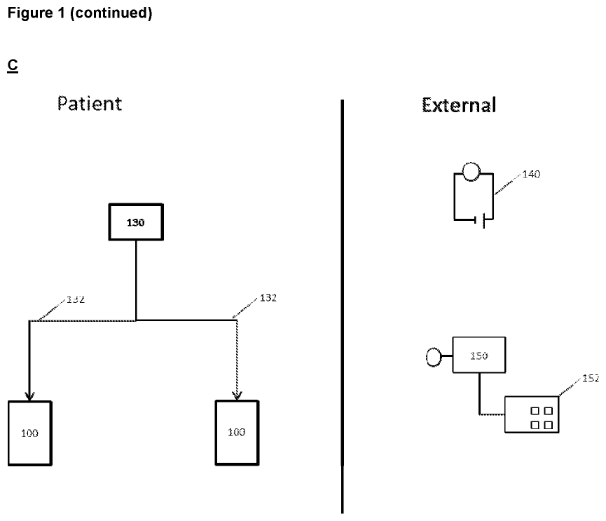

Implementation of all aspects of the present disclosure (as discussed both above and below) will be further appreciated by reference to FIGS. 1A-1C.

FIGS. 1A-1C show how the invention may be put into effect using one or more apparatuses which are implanted in, located on, or otherwise disposed with respect to a subject in order to carry out any of the various methods described herein. In this way, one or more apparatuses can be used to treat bladder dysfunction in a subject, by stimulating neural activity in a pudendal nerve.

In FIG. 1A a separate apparatus 100 is provided for unilateral neuromodulation, although as discussed above and below an apparatus could be provided for bilateral neuromodulation (100, FIGS. 1B and 1C). Each such apparatus may be fully or partially implanted in the subject, or otherwise located, so as to provide neuromodulation of the respective nerve or nerves. FIG. 1A also schematically shows in the cutaway components of one of the apparatuses 100, in which the apparatus comprises several elements, components or functions grouped together in a single unit and implanted in the subject. A first such element is an electrode 102, which is shown in proximity to a pudendal nerve 90 of the subject. The apparatus may optionally further comprise further electrodes (not shown) implanted proximally to the same or other pudendal nerve. Alternatively, the other pudendal nerve may be provided with a separate apparatus 100 (not shown). The primary electrode 102 may be operated by a controller 104. The apparatus may comprise one or more further elements such as a communication element 106, a detector 108, a power supply element 110 and so forth. Each apparatus 100 may operate independently, or may operate in communication with each other, for example using respective communication elements 106.

Each neuromodulation apparatus 100 may carry out the required stimulation in response to one or more control signals. Such a control signal may be provided by the controller 104 according to an algorithm independently, in response to output of one or more detector elements 108, and/or in response to communications from one or more external sources (for example an input element) received using the communications element. As discussed herein, the detector(s) could be responsive to a variety of different physiological parameters.

FIG. 1B illustrates some ways in which the apparatus of FIG. 1A may be differently distributed. For example, in FIG. 1B the apparatuses 100 comprise electrodes 102 implanted proximally to a pudendal nerve 90, but other elements such as a controller 104, a communication element 106 and a power supply 110 are implemented in a separate control unit 130 which may also be implanted in, or carried by the subject. The control unit 130 then controls the electrodes in both of the apparatuses via connections 132 which may for example comprise electrical wires and/or optical fibres for delivering signals and/or power to the electrodes.

In the arrangement of FIG. 1B one or more detectors 108 are located separately from the control unit, although one or more such detectors could also or instead be located within the control unit 130 and/or in one or both of the apparatuses 100. The detectors may be used to detect one or more physiological parameters of the subject as described elsewhere herein, and the controller or control unit then causes the transducers to apply the first or second signal in response to the detected parameter(s), for example only when a detected physiological parameter meets or exceeds a predefined threshold value. Physiological parameters which could be detected for such purposes may be selected from nerve activity in the pudendal nerve, nerve activity in the hypogastric nerve, nerve activity in the pelvic nerve, muscle activity in the bladder detrusor muscle, muscle activity in the internal urethral sphincter, muscle activity in the external urethral sphincter, muscle activity in the external anal sphincter, and bladder pressure.

A variety of other ways in which the various functional elements could be located and grouped into the neuromodulation apparatuses, a control unit 130 and elsewhere are of course possible. For example, one or more sensors of FIG. 1B could be used in the arrangement of FIG. 1A or 1C or other arrangements.

FIG. 1C illustrates some ways in which some functionality of the apparatus of FIG. 1A or 1B is provided not implanted in the subject. For example, in FIG. 1C an external power supply 140 is provided which can provide power to implanted elements of the apparatus in ways familiar to the skilled person, and an external controller 150 provides part or all of the functionality of the controller 104, and/or provides other aspects of control of the apparatus, and/or provides data readout from the apparatus, and/or provides a data input element 152. The data input facility could be used by a subject or other operator in various ways, for example to input data relating to the behaviour of the subject and/or their desires (e.g. that they wish to delay onset of the voiding phase).

By way of further example, devices for stimulating nerve activity in the pudendal nerve are described in U.S. Pat. Nos. 7,571,000 and 8,396,555, each of which are incorporated herein by reference.

In a further aspect, the invention provides a method of treating bladder dysfunction in a subject comprising applying a first electrical signal to a pudendal nerve of the subject to stimulate activity in said pudendal nerve, wherein the first electrical signal comprises an AC waveform having a frequency in the range of from 0.1-100 Hz, optionally 0.1-50 Hz, and wherein the first electrical signal is applied in a burst pattern.

In other embodiments, application of the method is mediated using an apparatus according to the present disclosure.

In certain embodiments, the first electrical signal is applied to a pudendal nerve during a bladder filling phase, wherein application of said first electrical signal increases bladder capacity. In preferred such embodiments, the first electrical signal stimulates neural activity in sensory fibres of the pudendal nerve, optionally selectively stimulates neural activity in sensory fibres of the pudendal nerve, so as to produce an increase in bladder capacity.

In certain preferred embodiments, the first electrical signal is applied to a sensory pudendal nerve of the subject. In certain embodiments, the sensory pudendal nerve is the dorsal nerve of the penis/clitoris (DNP, also known as the dorsal genital nerve (DGN)).

In preferred such embodiments, application of the first electrical signal is started shortly prior to onset of voiding, for example immediately prior to voiding onset. In such embodiments, no signal is otherwise applied during the filling phase. Where the method is performed by an apparatus as described herein, such starting of the first electrical signal may be caused by imminent voiding being detected by the apparatus or may be caused by the subject indicating via an input element that they wish to delay voiding phase. Voiding onset can be determined by monitoring one or more indicators of imminent voiding, such as bladder pressure approaching a threshold indicative of imminent voiding, or EUS activity increasing to a level indicative of imminent voiding.

In certain embodiments, the first electrical signal is applied to a pudendal nerve at or shortly after the onset of a urine leakage, wherein application of said first electrical signal reduces the urine output. In preferred such embodiments, the first electrical signal stimulates neural activity in sensory fibres of the pudendal nerve, optionally selectively stimulates neural activity in sensory fibres of the pudendal nerve, so as to reduce unwanted urine output.

In certain embodiments, the first electrical signal is applied to a pudendal nerve during a bladder voiding phase, wherein application of said first electrical signal increases voiding efficiency. In preferred such embodiments, the first electrical signal stimulates neural activity in a compound pudendal nerve or the sacral root, so as to produce an increase in voiding efficiency.

In preferred such embodiments, the first electrical signal is applied during a voiding phase of the micturition cycle. Voiding may be determined by bladder detrusor muscle activity.

It is advantageous when looking to improve bladder function to be able to promote healthy function in a phase-specific manner. For example, it is desirable to promote improved bladder capacity and/or delayed voiding onset during filling, and also improve voiding efficiency during the voiding phase.

As noted above and demonstrated herein, bursting stimulation of a sensory pudendal nerve is able to promote bladder capacity, even when applied shortly before voiding onset. In addition, bursting stimulation of the compound pudendal nerve is shown to improve (e.g. promote) voiding efficiency. Sacral root (e.g. S1, S2, S3, or S4) bursting stimulation is expected to have a similar effect on voiding efficiency to compound pudendal nerve bursting stimulation. For instance, as shown in the Examples, bursting stimulation of the sacral root has similar effects on voiding efficiency to bursting stimulation of the compound pudendal nerve. It is further demonstrated herein that bursting stimulation of the pudendal motor nerve is able to promote voiding efficiency. This effect is previously reported in WO2017/066572.

Therefore, in certain embodiments where a first and a second signal are applied the stimulation results in a phase-specific improvement in bladder function—e.g. improved bladder capacity and/or delayed voiding onset during filling, and improved voiding efficiency during voiding.

In certain such embodiments, the first electrical signal is applied to a sensory pudendal nerve so as to increase bladder capacity in accordance with such embodiments provided herein, and the second electrical signal is applied to a compound pudendal nerve, a sacral root, or a pudendal motor nerve, so as to increase voiding efficiency in accordance with the embodiments provided herein.

In preferred such embodiments the first electrical signal is applied shortly prior to onset of voiding, for example immediately prior to voiding onset, and the second signal is applied during the voiding phase. In preferred such embodiments, no signal is otherwise applied during the filling phase.

The following embodiments are described in relation to the first electrical signal. However, in embodiments where a first signal and a second signal are applied, the embodiments apply equally and independently to the first and second signals.

In certain embodiments the first electrical signal has an amplitude in the range 0.05 to 10 T. In certain embodiments the first electrical signal has an amplitude in the range 0.1 to 10 T. The desired stimulation intensity (i.e. the desired multiple of threshold intensity “T”) can be achieved through controlled variation of the current or voltage of the signal, preferably the current.

In such embodiments, the first electrical signal has an amplitude in the range from 0.05 T to 5.0 T. In certain embodiments, the first signal has an amplitude in the range from 0.3 T to 3 T. In some embodiments, the first electrical signal has an amplitude of 0.05, 0.1, 0.2, 0.3, 0.4, 0.5 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 T.

In certain embodiments, the first electrical signal has an amplitude value in the range of from 0.5 T to 3 T, preferably in the range of from 1 T to 3 T. In certain embodiments, the first electrical signal has an amplitude value in the range of from 1 T to 2.2 T. In certain embodiments, the first electrical signal has an amplitude value of at least 1 T. In certain embodiments, the first electrical signal has an amplitude value of at least 1.2 T, optionally at least 1.3 T, optionally at least 1.4 T, optionally at least 1.5 T, optionally at least 1.6 T, optionally at least 1.7 T, optionally at least 1.8 T, optionally at least 1.9 T, optionally at least 2 T.

In some embodiments, the electrical signal has an amplitude of 1 T, 1.5 T, 1.8 T, 2 T, 2.3 T, 2.5 T or 3 T. In certain preferred embodiments the first electrical signal has an amplitude in the range of from 1.8 T to 3 T, preferably 1.8 T to 2.3 T. In certain preferred embodiments the first electrical signal has an amplitude in the range of from 2 T to 3 T.

In other embodiments, the first electrical signal has an amplitude of 1 T. In other embodiments, the first electrical signal has an amplitude of 1.5 T. In other embodiments the first electrical signal has an amplitude of 2 T. In other embodiments the first electrical signal has an amplitude of 3 T.

In other embodiments, the first electrical signal has an amplitude in the range of from 0.1-20 mA, optionally 0.1-10 mA, optionally 0.1-5 mA, optionally 0.1-1 mA, optionally 100-500 μA, optionally 100-400 μA. In certain embodiments, the first electrical signal has an amplitude of 100 μA, 200 μA, 300 μA, or 400 μA.

In certain embodiments, the burst pattern of the first electrical signal comprises a signal burst having a duration in the range of from 20 ms to 2000 ms, optionally in the range of from 20 ms to 500 ms, optionally a duration in the range of from 20 ms to 200 ms. In certain preferred embodiments the burst pattern of the first electrical signal comprises a signal burst having a duration in the range of from 20 ms to 100 ms, optionally in the range of from 50 ms to 100 ms. In certain preferred embodiments the burst pattern of the first electrical signal comprises a signal burst having a duration of 50 ms. In certain preferred embodiments the burst pattern of the first electrical signal comprises a signal burst having a duration of 100 ms.

In certain embodiments, the burst pattern of the first electrical signal comprises a signal burst repeated at an interval of from 0.1 to 2 s, optionally 0.125 s to 2 s. In certain embodiments, the burst pattern comprises a signal burst repeated at an interval of from 0.125 s to 1 s. In certain embodiments, the burst pattern comprises a signal burst repeated at an interval of from 0.125 s to 0.5 s. In certain embodiments, the burst pattern comprises a signal burst repeated at an interval of 0.125 s. In certain embodiments, the burst pattern comprises a signal burst repeated at an interval 0.2 s. In certain embodiments, the burst pattern comprises a signal burst repeated at an interval of 0.5 s.

In certain embodiments, the burst pattern of the first electrical signal comprises a signal burst having a duration from 20 ms to 2000 ms repeated at an interval of from 0.1 s to 1 s. In certain embodiments, the burst pattern comprises a signal burst having a duration from 50 ms to 1000 ms repeated at an interval of from 0.125 s to 0.5 s.

In certain embodiments the burst pattern of the first electrical signal comprises a signal burst having a duration of 50 ms repeated at an interval of 0.5 s, optionally wherein the burst pattern has a signal burst having a duration of 50 ms repeated at an interval of 0.5 s.

In certain embodiments the burst pattern of the first electrical signal comprises a signal burst having a duration of 50 ms repeated at an interval of 0.2 s, optionally wherein the burst pattern has a signal burst having a duration of 50 ms repeated at an interval of 0.2 s.

In certain embodiments the burst pattern of the first electrical signal comprises a signal burst having a duration of 50 ms repeated at an interval of 0.125 s, optionally wherein the burst pattern has a signal burst having a duration of 50 ms repeated at an interval of 0.125 s.

In certain embodiments the burst pattern of the first electrical signal comprises a signal burst having a duration of 100 ms repeated at an interval of 0.5 s, optionally wherein the burst pattern has a signal burst having a duration of 100 ms repeated at an interval of 0.5 s.

In certain embodiments the burst pattern of the first electrical signal comprises a signal burst having a duration of 100 ms repeated at an interval of 0.2 s, optionally wherein the burst pattern has a signal burst having a duration of 100 ms repeated at an interval of 0.2 s.

In certain embodiments the burst pattern of the first electrical signal comprises a signal burst having a duration of 100 ms repeated at an interval of 0.125 s, optionally wherein the burst pattern has a signal burst having a duration of 100 ms repeated at an interval of 0.125 s.

In some embodiments, the first electrical signal applied in a burst pattern comprises a signal burst repeated at a frequency in the range of from 0.5 to 20 Hz. In some embodiments, the first electrical signal applied in a burst pattern comprises a signal burst repeated at a frequency in the range of 1 to 20 Hz. In some embodiments, the first electrical signal applied in a burst pattern comprises a signal burst repeated at a frequency in the range of 2 to 10 Hz, preferably in the range of from 2 Hz to 8 Hz.

In one embodiment, the first electrical signal comprises a signal burst repeated at a frequency comprising 10 Hz, optionally wherein the first electrical signal comprises an AC waveform repeated at a frequency of 10 Hz. In another embodiment, the first electrical signal is repeated at a frequency comprising 2 Hz, optionally wherein the first electrical signal is repeated at a frequency of 2 Hz. In another embodiment, the first electrical signal is repeated at a frequency comprising 4.76 Hz, optionally wherein the first electric signal is repeated at a frequency of 4.76 Hz. In another embodiment, the first electrical signal is repeated at a frequency comprising 8 Hz, optionally wherein the first electric signal is repeated at a frequency of 8 Hz.

In certain embodiments, the first electrical signal comprises a signal burst wherein the signal burst comprises an AC waveform having a frequency in the range of from 10-100 Hz. In certain embodiments, the first electrical signal comprises a signal burst wherein the signal burst comprises an AC waveform having a frequency in the range of from 0.1-50 Hz. In certain embodiments, the first electrical signal comprises a signal burst wherein the signal burst comprises an AC waveform having a frequency in the range of from 20-50 Hz.

In certain embodiments, the signal burst comprises an AC waveform having a frequency in the range of from 30-50 Hz, optionally in the range of from 30-40 Hz. In certain such embodiments, the signal burst has a frequency of 40 Hz.

In some embodiments, the burst pattern consists of from 1 to 10 pulses per signal burst. In some embodiments, the burst pattern consists of from 1 to 5 pulses per signal burst. In some embodiments, the burst pattern consists of 3 or 4 pulses per signal burst. In such embodiments, the duration of the signal burst is thus determined by the frequency of the AC waveform.

It will be appreciated by the skilled person that an electronic signal “comprising” an indicated frequency may have other frequency components as part of the signal. In certain preferred embodiments of all aspects where a signal comprises an indicated frequency, the indicated frequency is the dominant frequency component of the signal.

In certain embodiments, the AC waveform is a biphasic waveform, optionally a charge-balanced biphasic waveform. In certain such embodiments, the waveform may be symmetrical or asymmetrical. In certain such embodiments, each phase of the biphasic waveform has a phase duration from 0.005 ms to 2 ms, optionally 0.01 to 1 ms, optionally 0.05 to 0.5 ms, optionally 0.05 to 0.2 ms, optionally 0.1 ms. In certain embodiments, each phase of a biphasic waveform is of equal duration. In certain alternative embodiments, each phase is of a different duration.

The AC waveform may be selected from pulsatile, sinusoidal, triangular, rectangular, square or a complex waveform.

In certain embodiments, treatment of bladder dysfunction, for example overactive bladder, may be characterised by an increase in bladder capacity during filling periods, greater control of the timing of voiding onset, an increase in voiding efficiency for voiding periods, or any combination thereof.

In certain embodiments, the method is a method for treating overactive bladder, neurogenic bladder, underactive bladder (UAB), urinary retention, or detrusor hyperactivity with impaired contractility (DHIC).

In certain embodiments the method further comprises detecting one or more physiological parameters in the subject to determine the ongoing phase of the micturition cycle in the subject, optionally wherein the one or more physiological parameters are selected from: nerve activity in the pudendal nerve, nerve activity in the hypogastric nerve, nerve activity in the pelvic nerve, muscle activity in the bladder detrusor muscle, muscle activity in the internal urethral sphincter, muscle activity in the external urethral sphincter, muscle activity in the external anal sphincter, and bladder pressure. For example, if the ongoing phase is the filling phase, this may be determined by detecting a steady increase in bladder pressure. By way of further example, it may be determined that it is shortly before onset of voiding by an increase in EUS activity. By way of still further example, voiding phase may be determined by detecting activity in bladder detrusor muscle.

In a further aspect the invention provides a neuromodulation system, the system comprising a plurality of apparatuses according to the invention. In such a system, each apparatus may be arranged to communicate with at least one other apparatus, optionally all apparatuses in the system. In certain embodiments, the system is arranged such that, in use, the apparatuses are positioned to bilaterally stimulate the pudendal nerves of a patient.

In such embodiments, the system may further comprise additional components arranged to communicate with the apparatuses of the system, for example a processor, a data input facility, and/or a data display module. In certain such embodiments, the system further comprises a processor. In certain such embodiments, the processor is comprised within a mobile device (for example a smart phone) or computer.

In a further aspect, the invention provides a pharmaceutical composition comprising a compound for treating bladder dysfunction, for use in a method of treating bladder dysfunction in a subject, wherein the method is a method according to the invention, the method further comprising the step of administering an effective amount of the pharmaceutical composition to the subject. It is a preferred embodiment that the pharmaceutical composition is for use in a method of treating bladder dysfunction wherein the method comprises applying a signal to a part or all of a pudendal nerve of said patient to stimulate the neural activity of said nerve in the patient, the signal being applied by a neuromodulation apparatus as provided herein.

In a further aspect, the invention provides a pharmaceutical composition comprising a compound for treating bladder dysfunction, for use in treating bladder dysfunction in a subject, the subject having an apparatus according to the invention implanted. That is, the pharmaceutical composition is for use in treating a subject that has had an apparatus as described according to the first aspect implanted. The skilled person will appreciate that the apparatus has been implanted in a manner suitable for the apparatus to operate as described. Use of such a pharmaceutical composition in a patient having an apparatus according to the first aspect implanted will be particularly effective as it permits a cumulative or synergistic effect as a result of the combination of the compound for treating bladder dysfunction and apparatus operating in combination.

In certain embodiments of these aspects, the compound for treating bladder dysfunction is selected from an antimuscarinic compound and a β-adrenergic receptor agonist, optionally a β3-adrenergic receptor agonist. In certain embodiments, the antimuscarinic compound is selected from darifenacin, hyoscyamine, oxybutynin, tolterodine, solifenacin, trospium, or fesoterodine. In certain embodiments, the β-adrenergic receptor agonist is a β3-adrenergic receptor agonist, for example mirabegron. In certain embodiments, the pharmaceutical composition is for use in treating OAB.

In certain embodiments, the pharmaceutical composition may comprise a pharmaceutical carrier and, dispersed therein, a therapeutically effective amount of the compounds for treating bladder dysfunction. The composition may be solid or liquid. The pharmaceutical carrier is generally chosen based on the type of administration being used and the pharmaceutical carrier may for example be solid or liquid. The compounds of the invention may be in the same phase or in a different phase than the pharmaceutical carrier.

Pharmaceutical compositions may be formulated according to their particular use and purpose by mixing, for example, excipient, binding agent, lubricant, disintegrating agent, coating material, emulsifier, suspending agent, solvent, stabilizer, absorption enhancer and/or ointment base. The composition may be suitable for oral, injectable, rectal or topical administration.

For example, the pharmaceutical composition may be administered orally, such as in the form of tablets, coated tablets, hard or soft gelatine capsules, solutions, emulsions, or suspensions. Administration can also be carried out rectally, for example using suppositories, locally or percutaneously, for example using ointments, creams, gels or solution, or parenterally, for example using injectable solutions.

For the preparation of tablets, coated tablets or hard gelatine capsules, the compounds for treating bladder dysfunction may be admixed with pharmaceutically inert, inorganic or organic excipients. Examples of suitable excipients include lactose, maize starch or derivatives thereof, talc or stearic acid or salts thereof. Suitable excipients for use with soft gelatine capsules include, for example, vegetable oils, waxes, fats and semi-solid or liquid polyols.

For the preparation of solutions and syrups, excipients include, for example, water, polyols, saccharose, invert sugar and glucose. For injectable solutions, excipients include, for example, water, alcohols, polyols, glycerine and vegetable oil. For suppositories and for local and percutaneous application, excipients include, for example, natural or hardened oils, waxes, fats and semi-solid or liquid polyols.

The pharmaceutical compositions may also contain preserving agents, solublizing agents, stabilizing agents, wetting agents, emulsifiers, sweeteners, colorants, odorants, buffers, coating agents and/or antioxidants.

Thus, a pharmaceutical formulation for oral administration may, for example, be granule, tablet, sugar coated tablet, capsule, pill, suspension or emulsion. For parenteral injection for, for example, intravenous, intramuscular or subcutaneous use, a sterile aqueous solution may be provided that may contain other substances including, for example, salts and/or glucose to make to solution isotonic. The compound may also be administered in the form of a suppository or pessary or may be applied topically in the form of a lotion, solution, cream, ointment or dusting powder.

In a preferred embodiment of all aspects of the invention, the subject or patient is a mammal, more preferably a human. In certain embodiments, the subject or patient is suffering from bladder dysfunction, for example OAB.

The foregoing detailed description has been provided by way of explanation and illustration and is not intended to limit the scope of the appended claims. Many variations in the presently preferred embodiments illustrated herein will be apparent to one of ordinary skill in the art and remain within the scope of the appended claims and their equivalents.

### EXAMPLES

The invention will be further understood with reference to the following non-limiting examples.

### Example 1. Bursting Stimulation Applied to the Pudendal Sensory Branch During Voiding

Bladder function is comprised of two phases: a filling phase (urine storage) and a voiding phase (micturition). Efficient bladder function involves control of these phases mediated by continence and micturition reflexes accomplished through coordinated sympathetic, parasympathetic and somatic neural activity modulated at the level of the spinal cord. The pudendal nerve and its branches have a critical role in both the guarding-reflex to promote continence and to also facilitate micturition via the voiding-reflex. In bladder dysfunction and conditions such as over-active bladder (OAB) or underactive bladder (UAB), one or more of these functions are disrupted. Selective neuromodulation of the pudendal nerve branches during the filling or voiding phase provides an opportunity to positively impact bladder dysfunction.

Several experiments were conducted to assess the effects of bursting stimulation on different components of the pudendal nerve. In a first experiment, bursting stimulation was applied to the pudendal sensory branch (DGN) of male and female cats as 2 Hz bursts, 40 Hz, 50 ms duration (3 pulses per burst).

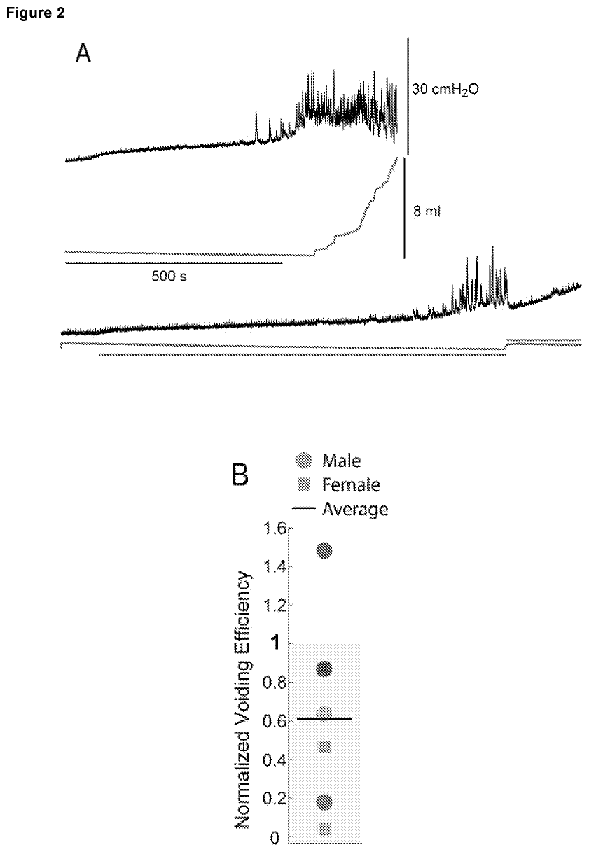

The results of example cystometrograms from a female cat are depicted in FIG. 2A. The upper trace shows control bladder pressure and voiding in no-stimulation conditions. In the lower trace, continuous pudendal sensory branch stimulation during the filling phase increased bladder capacity, and application of bursting stimulation to the same site at void onset inhibited ongoing bladder contractions and stopped urine output.

FIG. 2B summarizes the effect of pudendal sensory branch bursting stimulation on voiding efficiency in cats (male n=4; female n=2) following stimulation during bladder filling on the same nerve. Continuous stimulation during filling (10 Hz, 3 T) increased bladder capacity, and application of bursting stimulation during voiding caused a reduction in voiding efficiency. Except for one male cat, pudendal sensory branch bursting increased continence, as indicated by a decrease in voiding efficiency relative to controls. This demonstrates that pudendal sensory bursting during voiding is able to reduce unwanted urine output.

### Example 2. Burstinq Stimulation of the Sensory Pudendal Nerve in the Female Rat

The effect of bursting stimulation of the sensory pudendal nerve was further investigated in a rat model.

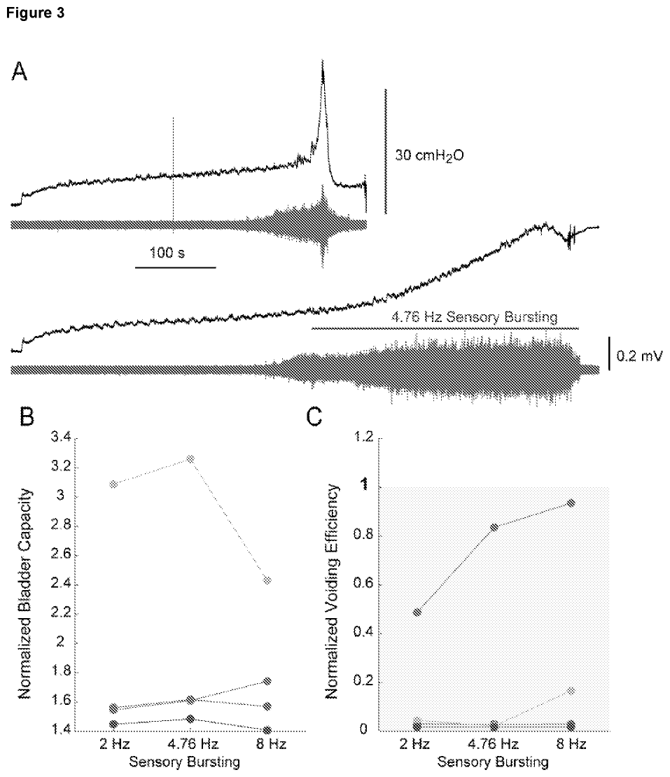

FIG. 3A shows example cystometrograms consisting of a no-stimulation trial and a stimulation trial that included sensory pudendal bursting at a 4.76 Hz train rate. The onset of stimulation occurred at the volume that was expected just before the bladder contraction in the previous control trial (top trace). Bursting stimulation inhibited bladder contractions and eventually the trial was stopped after urine leaked at a high bladder pressure.

The signal bursts of the stimulation signal were applied at different frequencies (2 Hz, 4.76 Hz, and 8 Hz, each burst at 40 Hz, 1 T or 1.5 T) and the results compared, as shown in FIG. 3B. Sensory bursting increased bladder capacity by at least 40% as well as producing carry over effects. Unlike the protocol in the cat, there was no stimulus during the filling phase. Stimulation was only conducted just prior to the volume at which a bladder contraction was expected (based on preceding control trials). Nevertheless, sensory bursting at all burst frequencies resulted in increased bladder capacity.

Sensory bursting at 1 T using different train rates during the voiding phase decreased voiding efficiency relative to controls. In no experiments did the voiding efficiency exceed control levels, again indicating that bursting stimulation applied to the pudendal sensory branch during voiding promotes continence.

### Example 3. Burstinq Stimulation on the Compound Pudendal Nerve During Voiding

The effect of bursting stimulation of the compound pudendal nerve was also assessed in a cat model.

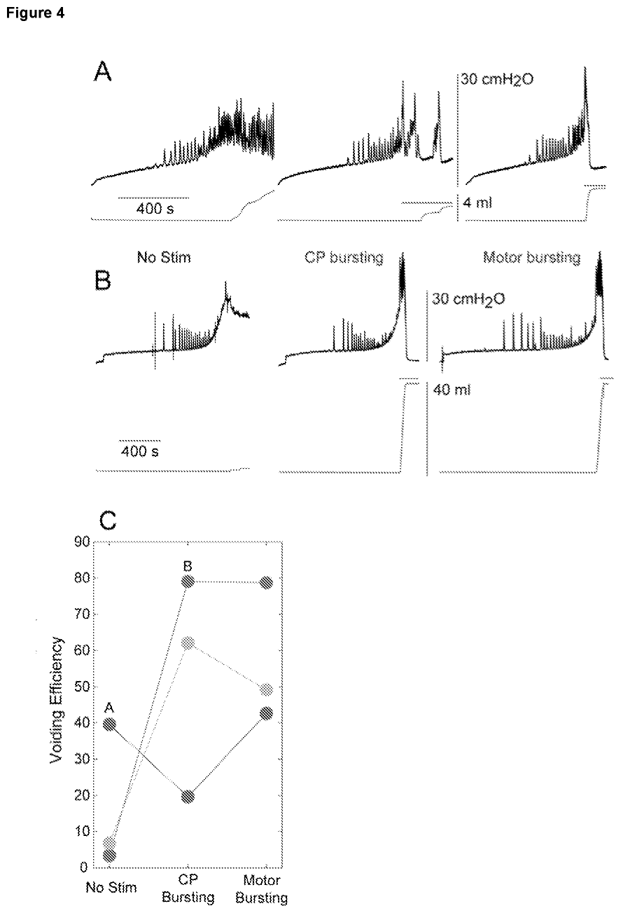

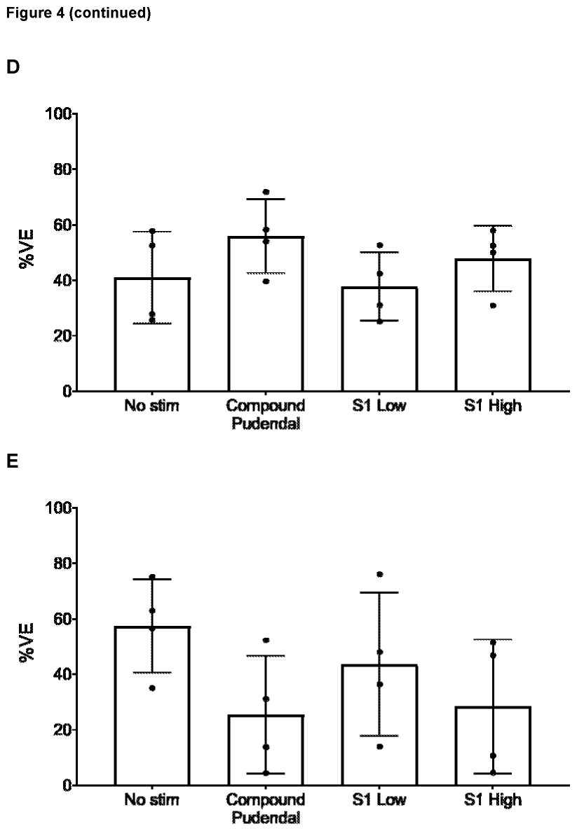

FIG. 4A shows example cystometrograms from one experiment, with a no-stimulation trial (left), compound pudendal nerve bursting (middle), and pudendal motor branch bursting (right, as described in WO2017/066572). Here compound pudendal nerve stimulation generated coordinated bladder contractions, though urine output was low compared to the other conditions.

In a second experiment (FIG. 4B) with the same layout as FIG. 4A, compound pudendal nerve bursting stimulation led to a coordinated bladder contraction and higher urine output. Compound pudendal nerve stimulation and pudendal motor stimulation resulted in similar voiding efficiency values.

The effect of compound pudendal nerve bursting stimulation was therefore further investigated. FIG. 4C displays the voiding efficiency values from three experiments, comparing the effect of no-stimulation during voiding against bursting stimulation on the compound pudendal nerve or pudendal motor branch intervention sites. For the two experiments in which compound pudendal nerve stimulation increased voiding efficiency, the voiding efficiency increased from below 10% to above 60%.

FIG. 4D confirms that compound pudendal nerve bursting stimulation of the compound pudendal nerve during voiding results in an increase in voiding efficiency in female subjects relative to controls. Similar effects were observed when bursting stimulation was applied to the sacral root at a high amplitude (≥2 T−S1 high) but not when applied at a low amplitude (0.9 T−S1 low).

FIG. 4E shows that bursting stimulation of the compound pudendal nerve in male subjects during voiding results may improve continence. Similar results were observed when bursting stimulation was applied to the sacral root at a high amplitude (≥2 T−S1 high) but not when applied at a low amplitude (0.9 T−S1 low).

Amplitude T was determined for sacral root stimulation by reference to the EMG response in the gastrocnemius muscle, where 1 T was the minimum amplitude that evoked EMG activity.

Although sacral root S1 was used in the cat experiments, this can be considered representative of suitable stimulation of the sacral root in humans, for example at S2, S3, or S4.

### CONCLUSION

The results provided herein demonstrate that the pudendal bladder reflex pathways can be altered through neuromodulation, and that bladder capacity and/or voiding efficiency can be improved by pudendal nerve stimulation, for example by selective stimulation of the compound pudendal nerve, pudendal sensory branches or motor branches. This therapeutically enables the targeting of differing bladder dysfunction conditions: OAB to increase bladder capacity, or UAB to promote efficient voiding and reduce post void residual volumes.

### Methods

### Rat Surgical Preparation and Procedures

Female Wistar rats (n=4) weighing between 238 and 257 g were anesthetized with urethane (1.2 g/kg SC, supplemented as necessary). Body temperature was monitored using an esophageal temperature probe and maintained at 36-38° C. with a water blanket. Heart rate and arterial blood oxygen saturation levels were monitored using a pulse oximeter (Nonin Medical Inc., 2500A VET).

In preparation for cystometrogram (CMG) measurements, the bladder was exposed via a midline abdominal incision. The tip of a polyethylene (PE-90) catheter (Clay Adams, Parsippany, N.J) was heated to create a collar and inserted into the bladder lumen through a small incision in the apex of the bladder dome which was secured with a 6-0 silk suture. The abdominal wall was closed in layers with 3-0 silk suture. The bladder catheter was connected via a 3-way stopcock to an infusion pump (Braintree Scientific Inc., BS-8000 or Harvard Apparatus PHD 4400) and to a pressure transducer (ArgoTrans, ArgonMedical Devices Inc., Plano, Tex.) connected to a bridge amplifier and filter (13-6615-50, Gould Instruments, Valley View, Ohio) for measuring intravesical pressure (IVP). Data were sampled at 1 kHz using a PowerLab system (AD Instruments, Colorado Springs, Colo.).

External urethral sphincter (EUS) EMG was measured using two platinum contacts bonded to a silicone backing with wires welded to each contact (Microleads, Boston). This sheet-electrode was placed intra-abdominally between the urethra and the pubic symphysis (Hokanson et al., 2017b). EUS EMG leads were connected through a preamplifier (HIPS, Grass Products, Warwick, R.I.) to an amplifier (P511, Grass Products). A subcutaneous needle served as ground. Signals were filtered (3 Hz-3 kHz) and sampled at 20 kHz.

After placing the bladder catheter and EUS EMG electrodes, the animal was turned to a prone position for cuff placement. After resecting gluteal muscles at the midline, the ischium was spread apart from the sacrum to expose the ischiorectal fossa, and the sensory branch of the pudendal nerve was isolated from connective tissue. A 200 μm inner diameter (2 mm length) was placed on the pudendal sensory branch (CorTec, Freiburg, Germany). Following nerve cuff placement, the incision was sutured closed in layers with 3-0 silk suture. The animal was then turned back into a supine position for cystometric testing.

### Rat Electrical Stimulation

Electrical stimulation was delivered using a stimulus generator (model STG4002-16 mA, Multi-Channel Systems, Reutlingen, Germany). Stimulation pulses consisted of a charge-balanced biphasic waveform with 100 μs pulse widths. Strength of stimulation was assessed by monitoring evoked EUS EMG. Amplitudes for sensory pudendal nerve stimulation were normalized to the minimum stimulation amplitude necessary to reflexively evoke EUS EMG activity. This stimulation amplitude is referred to as 1 T (1 times threshold amplitude).

Electrical stimulation was initiated just prior to bladder voiding or urine leakage and continued throughout the bladder contraction. Differing bursting stimulation paradigms were compared, consisting of 3 pulses at 40 Hz repeated at either 2 Hz (every 0.5 seconds), 4.76 Hz (every 0.21 seconds), or 8 Hz (every 0.125 seconds). The stimulation amplitude used for these experiments was 1 T or 1.5 T. Results from 1 and 1.5 T were similar and were merged for analysis.

### Cat Surgical Preparation and Procedures

Acute experiments were conducted in adult neurologically intact male (n=4, 3.5-3.8 kg) and female (n=2, 3 and 3.2 kg) cats. Anesthesia was induced with isoflurane (3%) and maintained with α-chloralose (65 mg/kg initial dose followed by continuous infusion of 5 mg/kg/h iv and supplemented as necessary based on jaw tone and blood pressure) following completion of the surgery. Gentamicin 5 mg IM and ketofen 1.2 g/kg SQ were given prior to surgical incision. A tracheotomy was performed to place a silicone endotube (Cat. no J0612B, Jorgensen Laboratories, Loveland, Colo.) connected to an artificial respirator (ADS 1000, Engler Engineering Corporation, Hialeah, Fla.), and artificial respiration was controlled to maintain end-tidal CO₂ between 3-4% (Capnoguard, Novametrix Medical Systems Inc., Wallingford, Conn.). The right carotid artery was cannulated with a 3.5 Fr polypropylene catheter (Cat. no 8890703211, Medtronic, Minneapolis, Minn.) to monitor arterial blood pressure (Tektronix 413A Neonatal Monitor) and was kept patent by infusing saline at a constant rate of 6 ml/hr. Body temperature was measured using an esophageal temperature probe and maintained at 38° C. with a forced-air warming blanket (Bair Hugger model 505, 3M). Additional fluids (0.9% physiological saline with 5% dextrose and 8.4 g/L NaHCO₃) were administered continuously (15 ml/kg/hr, i.v.) via the left cephalic catheter. Following a midline abdominal incision, the bladder was cannulated through the dome with a modified 14 g BD Angiocath catheter connected to PE 90 tubing introduced with a hypodermic needle, secured with a purse string suture (4-0 silk, Cat. No M-S418R19, AD Surgical, Sunnyvale, Calif.) and connected to a solid-state pressure transducer (Deltran, Utah Medical, UT) to measure bladder pressure. A force transducer (model: MLT500D, AD Instruments, Colorado Springs, Colo.) was used to collect voided volume (VV). The external anal sphincter (EAS) EMG activity was measured by PFA-coated platinum-iridium wires (0.0055 inch-diameter, A-M Systems, Sequim, Wash.) inserted percutaneously into the EAS bilaterally. EAS EMG leads were connected through a preamplifier (HIPS, Grass Products, Warwick, R.I.) to an amplifier (P511, Grass Products). Bladder pressure (BP), VV, and EAS EMG signals were amplified, filtered, and sampled at either 1,000 Hz (BP and VV) or 20,000 Hz (EAS EMG). The gastrocnemius muscle (gastroc) EMG activity was measured by PFA-coated platinum-iridium wires (0.0055 inch-diameter, A-M Systems, Sequim, Wash.) inserted percutaneously into the gastric ipsilateral to stimulation. Gastrocnemius EMG leads were connected through a preamplifier (HIPS, Grass Products, Warwick, R.I.) to an amplifier (P511, Grass Products). Gastrocnemius EMG signals were amplified, filtered, and sampled at 20,000 Hz.

For experiments assessing the pudendal nerves for stimulation, the common pudendal nerve was exposed through an incision between the base of the tail and the ischial tuberosity, transection of the gluteofemoralis, and dissection of the ischiorectal fossa. The pudendal sensory nerve and pudendal motor branches are visible with further dissection of the distal nerve portions. A bipolar cuff electrode (300-500 μM, CorTec: depending on animal nerve size) was placed on the differing intervention sites, i.e. common pudendal, pudendal sensory nerve (dorsal genital neve), and/or pudendal motor branch. For experiments assessing sacral stimulation, the L6-S1 vertebra is exposed, and a partial laminectomy of the L6 vertebra performed to expose the spinal cord and DRG's. An octa-polar lead was inserted into the space between the cord and vertebra near the S1 DRG (S1 feline homologue of human S3). To confirm/adjust the stimulation lead, stimulation at 1 Hz was conducted to verify the presence of EUS reflex. If this was not observed, the lead was slowly moved until the reflex became visible. The amplitude threshold (“T”) was confirmed as the level required to evoke reflex activity in the EMG signal at 1 Hz.

The bladder was continuously filled with physiological saline at room temperature (0.2-2.2 ml/min, median=1.1 ml/min) using an infusion pump (model: PHD 4400, Harvard Apparatus), with an open urethra for approximately one hour to allow post-surgical recovery. The bladder was subsequently emptied and cystometrograms (CMGs) recorded. For each CMG, the bladder was filled micturition or urine leakage occurred until, at which time the infusion pump was turned off. Approximately one minute after the bladder pressure returned to baseline, the bladder was emptied via the catheter using a syringe. Within a block of trials, the filling rate remained constant. Voided (VV) and residual (RV) volumes were recorded and used to calculate bladder capacity (BC) and voiding efficiency (VE).

### Cat Electrical Stimulation

Electrical stimulation was delivered using a stimulus generator (model STG4004-16 mA, Multi-Channel Systems). Stimulation pulses consisted of a charge-balanced biphasic waveform with 100 μs per phase. Strength of stimulation was assessed by monitoring evoked EAS EMG. For sensory/DGN and compound pudendal stimulation, stimulus threshold was defined as the stimulus amplitude necessary to evoke reflex EAS EMG activity.

Electrical stimulation to promote bladder filling started at filling onset. The stimulus consisted of sensory pudendal nerve stimulation (females) or DGN stimulation (males) at 3 T and 10 Hz. In one set of experiments, stimulation was switched to a bursting paradigm during the voiding phase. The bursting pattern consisted of 3 pulses at 40 Hz, 3 T, at a 2 Hz train rate on the same nerve (sensory/DGN).

In other experiments no stimulation was utilised during the bladder cystometry filling phase. At void onset, bursting stimulation was initiated being applied to the compound pudendal nerve or sacral root. The bursting pattern consisted of 3 pulses at 40 Hz at a 2 Hz train rate. For pudendal motor branch nerve stimulation, the minimum amplitude that evoked a maximal EAS EMG response was used. For compound pudendal stimulation amplitudes were varied from 1 T to 2.2 T, with results reported for 1.6 T, 2 T, and 2.2 T for the three experiments in FIG. 4. For sacral root stimulation amplitudes two levels were assessed: 1). “High” was the amplitude that produced maximal EUS or EAS EMG activation at 1 Hz, equivalent to at least 2 T, and 2). “Low” was the minimal amplitude that did not produce gastrocnemius muscle (gastroc) evoked EMG activity, equivalent to 0.9 T.

### Data Analysis

For each trial (cystometrogram) bladder capacity was calculated as the sum of the voided volume and the residual volume extracted from the bladder. Voiding efficiency was calculated as the ratio of the voided volume to the bladder capacity.

1. An apparatus for stimulating neural activity in a pudendal nerve of a subject, the apparatus comprising:
at least one primary electrode configured to apply a first electrical signal to said pudendal nerve; and
a controller coupled to said primary electrode(s) and controlling the first electrical signal to be applied thereby, wherein said controller is configured to cause said at least one primary electrode to apply said first electrical signal that stimulates neural activity in the pudendal nerve to improve bladder function, wherein the first electrical signal comprises an AC waveform having a frequency in a range of from 0.1-100 Hz and wherein the first electrical signal is applied in a burst pattern.

2.-4. (canceled)

5. The apparatus according to claim 1 in which the first electrical signal has an amplitude in the range of from 0.1-20 mA.

6. The apparatus according to claim 1 wherein the burst pattern of the first electrical signal comprises a signal burst having a duration in a range of from 20 ms to 2000 ms.

7. (canceled)

8. The apparatus according to claim 6 in which the burst pattern comprises a signal burst repeated at an interval of from 0.1 s to 2 s.

9.-16. (canceled)

17. The apparatus according to claim 1 in which the first electrical signal is to be applied to a sensory branch of the pudendal nerve.

18.-19. (canceled)

20. The apparatus according to claim 1 wherein the first electrical signal is to be applied to the pudendal nerve or sacral root.

21.-22. (canceled)

23. The apparatus according to claim 1, wherein the apparatus further comprises at least one secondary electrode configured to apply a second electrical signal to a compound pudendal nerve, a sacral root, or a pudendal motor nerve; and a controller coupled to said secondary electrode(s) and controlling the second electrical signal to be applied thereby, wherein said controller is configured to cause said at least one secondary electrode to apply said second electrical signal that stimulates neural activity in the compound pudendal nerve, sacral root, or pudendal motor nerve to improve voiding efficiency, wherein the second electrical signal comprises an AC waveform having a frequency in a range of from 0.1-100 Hz and wherein the second electrical signal is applied in a burst pattern, optionally wherein an improvement in voiding efficiency is an increase in voiding efficiency.

24.-41. (canceled)

42. A method of treating bladder dysfunction in a subject comprising:
i. implanting in the subject an apparatus according to claim 1;
ii. positioning at least one primary electrode of the apparatus in signalling contact with a pudendal nerve of the subject; and
iii. activating the apparatus to apply an electrical signal to the pudendal nerve of the subject as caused by the controller.

43. (canceled)

44. A method of treating bladder dysfunction in a subject comprising applying a first electrical signal to a pudendal nerve of the subject to stimulate activity in said pudendal nerve, wherein the first electrical signal comprises an AC waveform having a frequency in a range of from 0.1-100 Hz and wherein the first electrical signal is applied in a burst pattern.

45.-48. (canceled)

49. A method according to claim 44 in which the first electrical signal has an amplitude in a range of from 0.1-20 mA.

50. A method according to claim 44 wherein the burst pattern of the first electrical signal comprises a signal burst having a duration in a range of from 20 ms to 2000 ms.

51. (canceled)

52. A method according to claim 44 in which the burst pattern comprises a signal burst repeated at an interval of from 0.1 s to 2 s.

53.-60. (canceled)

61. A method according to claim 44 in which the first electrical signal is applied to a sensory branch of the pudendal nerve.

62. A method according to claim 44 in which the first electrical signal is applied to sensory fibres or a sensory branch of the pudendal nerve and stimulates neural activity in the sensory branch of the pudendal nerve to produce an increase in bladder capacity and/or a decrease in unwanted urine output.

63. (canceled)

64. A method according to claim 44 wherein the first electrical signal is to be applied to the pudendal nerve or sacral root.

65.-84. (canceled)

85. A method according to claim 44 further comprising detecting one or more physiological parameters in the subject to determine an ongoing phase of a micturition cycle in the subject, optionally wherein the one or more physiological parameters are selected from: nerve activity in the pudendal nerve, nerve activity in a hypogastric nerve, nerve activity in a pelvic nerve, muscle activity in a bladder detrusor muscle, muscle activity in an internal urethral sphincter, muscle activity in an external urethral sphincter, muscle activity in an external anal sphincter, and bladder pressure.

86. (canceled)

87. A method according to claim 44 wherein the first electrical signal is applied on receiving an input from the subject that the subject wishes to delay onset of a voiding phase.

88. A method according to claim 44 wherein the first electrical signal is applied on receiving an input from the subject indicating that the subject has sensed a urinary leak.

89. A method according to claim 44 wherein the first electrical signal is applied on receiving an input from the subject that the subject wishes to increase voiding efficiency.

90.-92. (canceled)

93. A neuromodulation system comprising a plurality of apparatuses according to claim 1.
